(19) United States
(12) Patent Application Publication
(10) Pub. No.: US 2005/0214373 A1
(43) Pub. Date: Sep. 29, 2005

(54) Coated tablet formulation and method

(76) Inventors: Divyakant S. Desai, West Windsor, NJ (US); Danping Li, East Brunswick, NJ (US)

Correspondence Address: STEPHEN B. DAVIS;BRISTOL-MYERS SQUIBB COMPANY, PATENT DEPARTMENT, P O BOX 4000, PRINCETON, NJ, 08543-4000, US

(21) Appl. No.: 11/085,710

(22) Filed: Mar. 21, 2005

Related U.S. Application Data
(60) Provisional application No. 60/556,331, filed on Mar. 25, 2004.
(60) Provisional application No. 60/648,872, filed on Feb. 1, 2005.

(51) Int. Cl. A61K 31/421; A61K 9/24; A61K 9/36

(52) U.S. Cl. 424/472; 514/374

(57) ABSTRACT

A coated tablet formulation is provided which includes a medicament such as the PPAR α/γ dual agonist peliglitazar or muraglitazar. The coated tablet includes a tablet core containing one or more fillers, one or more binders, one or more disintegrants, and other conventional excipients, and a coating on the tablet core, which coating may include one or more layers, at least one layer of which is formed of medicament and one or more coating polymers, preferably a hydroxypropylmethyl cellulose based polymer. A method for forming the coated tablet via a spray-dried coating technique is also provided.

This application claims a benefit of priority from U.S. Provisional Application Nos. 60/556,331, filed Mar. 25, 2004, and 60/648,872, filed Feb. 1, 2005, the entire disclosures of which are herein incorporated by reference.

### FIELD OF THE INVENTION

The present invention relates to a coated tablet formulation which includes a tablet core coated with a medicament such as a PPAR α/γ agonist, and to a method for preparing such coated tablet formulation.

### BACKGROUND OF THE INVENTION

The PPAR α/γ dual agonist having the structure 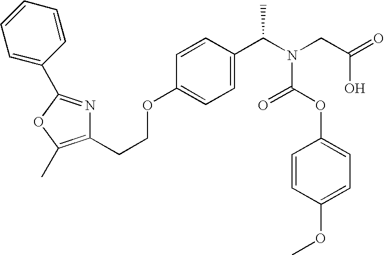
(generally referred to as peliglitazar) disclosed in U.S. Pat. No. 6,414,002, lowers glucose and lipid levels and thus is useful for the treatment of Type II diabetes and dyslipidemia. This compound has been found to undergo base catalyzed degradation and acid catalyzed degradation as shown below via the following reactions.

### Base Catalyzed Degradation of Compound A

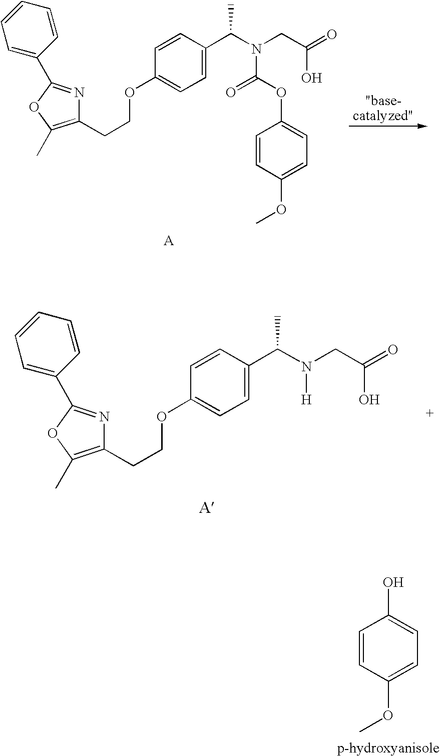

### Acid-Catalyzed Degradation of Compound A

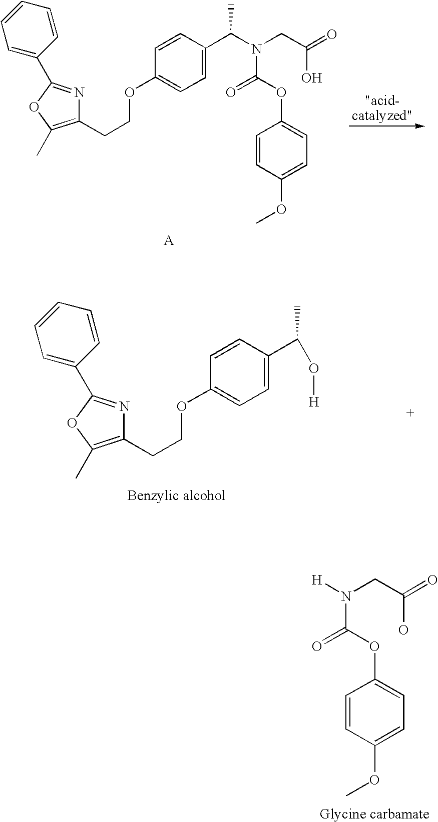

To avoid base catalyzed degradation, it has been suggested to add citric acid to a capsule formulation containing the PPAR α/γ dual agonist. However, it was found that the addition of citric acid did not prevent the formation of based catalyzed degradants completely. Moreover, there were acid catalyzed degradants as well. The level of degradation was unacceptable even at routine storage conditions of 25° C./60% relative humidity. Degradant formation of the capsule formulation was prevented only by refrigerating the capsules.

To circumvent the degradation problems associated with the capsule formulation, tablets were formulated as dry and wet granulation formulations, without adding any pH modifier such as citric acid. It was found that both dry and wet granulation formulations exhibited better stability to the capsule formulations and the wet granulated tablet exhibited superior stability to the dry granulated tablets. The dry granulated tablets continued to show presence of acid catalyzed degradants even without citric acid. The wet granulation tablets showed satisfactory stability at 30° C./60% relative humidity, but at accelerated conditions of 40° C./75% relative humidity (open) and 50° C. condition, there was a loss in potency accompanied by a large increase in degradation levels.

Thus, it is seen that there is clearly a need for stable pharmaceutical formulations containing medicaments which are subject to base catalyzed degradation and acid catalyzed degradation.

The PPAR α/γ dual agonist muraglitazar which has the structure 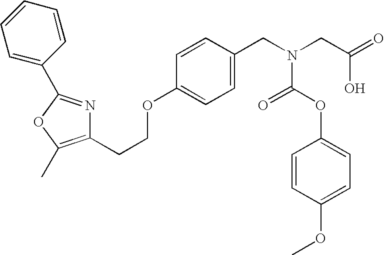
is also disclosed in U.S. Pat. No. 6,414,002.

### BRIEF DESCRIPTION OF THE INVENTION

In accordance with the present invention a coated tablet is provided which may include a medicament which is subject to base catalyzed degradation and/or acid catalyzed degradation, but is surprisingly stable under normal storage conditions, that is at 30° C. and 60% relative humidity.

The coated tablet of the invention includes a tablet core and at least one coating layer coated on the core, which coating layer is formed of a medicament and at least one coating polymer. The medicament will preferably be a compound covered by or disclosed in U.S. Pat. No. 6,414,002, including the PPAR α/γ dual agonist 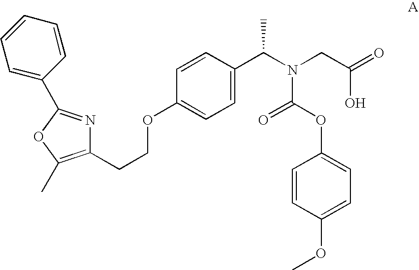
(also referred to as Compound A or peliglitazar) and the PPAR α/γ dual agonist 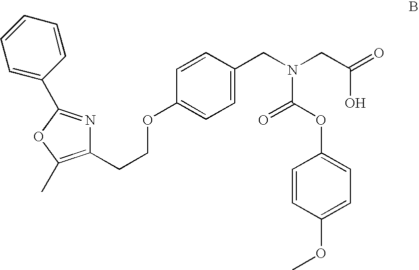
(also referred to as Compound B or muraglitazar).

In a preferred embodiment, the coated tablet of the invention will include a) a tablet core which is formed of one or more bulking agents or fillers, optionally one or more binders, optionally one or more disintegrants, and optionally one or more tableting lubricants, and optionally one or more medicaments, and b) at least one coating layer which includes one or more medicaments and coating polymer which is preferably a hydroxypropylmethyl cellulose based polymer, which coating layer is applied to the tablet core preferably by spray coating on to the tablet core.

The tablet core may be devoid of medicament or may include any medicament which may be employed in combination with the medicament in the coating layer. The medicament in the coating layer may be employed in the tablet core as well, although this is not preferred.

In a more preferred embodiment of the invention, a second coating layer will be coated over the initial coating layer (containing medicament) and will function as a protective layer. The second coating layer is preferably similar in composition to the initial coating layer except that it will not include a medicament. However, the second coating layer may also be formed of other coating polymers as well.

The coating layers are preferably applied by spray coating techniques.

It has been found that the coated tablets of the invention exhibit superior chemical stability as compared to traditional tablets manufactured using conventional dry granulation or wet granulation techniques. The spray coating approach involves only a single unit operation involving drug compared to five to six unit operations with traditional tableting methods. This is especially significant where the medicament requires special handling and therefore all unit operations need to be performed in a containment area. Moreover, less unit operations will reduce the cycle time. Where a medicament is employed which requires special handling, tablets containing such medicaments even when manufactured using traditional methods, such tablets will have to be coated to protect caregivers from such medicaments. The tablets are also coated to prevent photolytic degradation or hydrolysis of the drug in presence of moisture.

The spray coating approach will also facilitate preparation of a combination formulation of a problematic medicament with another drug by using the other drug tablet as a core tablet (instead of the tablet placebo core) and applying the spray coating containing the problematic medicament and coating polymer over the other drug tablet.

The coated tablets of the invention may be prepared using pan coaters or fluid-bed coating as well.

In addition, in accordance with the present invention, a method is provided for preparing the coated tablet of the invention, which method includes the steps of providing a tablet core and coating the tablet core with at least one coating layer formulation, and drying the coated tablet to form the coated tablet of the invention. The coating layer formulation includes a medicament and at least one coating polymer and a coating solvent.

In a preferred embodiment of the method of the invention the coating layer formulation is applied as a suspension of the coating polymer.

A second coating layer may be applied as a suspension over the dried first coating layer. The second coating layer need not include a medicament (although it may, if desired), and may be formed of the other components of the first coating layer.

In preparing the coated tablets of the invention, a coating suspension of medicament and coating polymer in water is prepared. Other coating solvents which may be employed include ethanol, methanol, and isopropyl alcohol, with water being preferred. Tablet cores (which preferably contain no medicament, medicament to be present in coating layer) are coated with the above suspension of medicament and coating polymer. The so-coated tablets are dried to produce the coated tablets of the invention.

Where the coated tablet of the invention is to include an outer protective layer, a coating suspension is prepared as in the case of the initial coating suspension but without medicament. The coating suspension will then be coated on to the previously coated tablets as described for the initial coating to form a protective coating layer thereon.

The coated tablets of the invention are useful in the treatment of mammals such as humans, dogs and cats for Type II diabetes and dyslypidemia.

### DETAILED DESCRIPTION OF THE INVENTION

The tablet core employed in the coated tablet of the invention will include conventional pharmaceutical excipients to enable formation of a pharmaceutically acceptable solid tablet core and optional medicaments. The tablet core may be in the form of a tablet, bead, beadlet, or pill, all of the above being collectively referred to as a tablet core.

The coated tablet of the invention will contain medicament, preferably a PPAR α/γ dual agonist as disclosed in U.S. Pat. No. 6,414,002 such as Compound A and Compound B, in an amount within the range from about 0.1% to about 70% by weight and preferably from about 0.25% to about 25% by weight of the finished tablet or from about 0.1 to about 200 mg, preferably from about 0.1 to about 50 mg, more preferably from about 0.1 to about 25 mg.

The tablet core employed in the coated tablet of the invention will preferably contain
a) at least one bulking agent or filler;
b) preferably but optionally at least one binder;
c) preferably but optionally at least one disintegrant;
d) preferably but optionally at least one lubricant; and
e) optionally at least one medicament;
   wherein
a) the bulking agent or filler is present in an amount within the range from about 1 to about 95% by weight, preferably from about 10 to about 85% by weight;
b) the binder is optionally present in an amount within the range from about 0 to about 20% by weight, preferably from about 1 to about 10% by weight;
c) the disintegrant is optionally present in an amount within the range from about 0 to about 20% by weight, and preferably from about 0.25 to about 15 % by weight;
d) the lubricant is optionally present in an amount within the range from about 0 to about 5% by weight, preferably from about 0.2 to about 2% by weight;
e) the optional medicament will be present in a therapeutic amount depending upon the nature of the medicament and/or as disclosed in the Physician's Desk Reference.

It is preferred that the bulking agents are microcrystalline cellulose and/or lactose monohydrate;
the disintegrant is croscarmellose sodium; and
the lubricant is magnesium stearate.

The tablet cores present in the coated tablets of this invention can be prepared by a variety of processes and order of addition of excipients. The utility of these formulations is not limited to a specific dosage form or manufacturing process. Tablet cores may be manufactured by wet granulation, dry granulation, direct blending or any other pharmaceutically acceptable process.

In accordance with the present invention, a preferred method is provided for preparing the tablet cores employed in the coated tablets of the invention which includes the steps of blending the one or more excipients such as bulking agent, disintegrant and lubricant , and compressing the blend into tablets. A lubricant will be preferably added to the blend to facilitate tablet compression.

The bulking agents or fillers will be present in the tablet compositions of the invention in an amount within the range from about 1 to about 95% by weight and preferably from about 10 to about 85% by weight of the composition. Examples of bulking agents or fillers suitable for use herein include, but are not limited to, cellulose derivatives such as microcrystalline cellulose or wood cellulose, lactose, sucrose, starch, pregelatinized starch, dextrose, mannitol, fructose, xylitol, sorbitol, corn starch, modified corn starch, inorganic salts such as calcium carbonate, calcium phosphate, dicalcium phosphate, calcium sulfate, dextrin/dextrates, maltodextrin, compressible sugars, and other known bulking agents or fillers, and/or mixtures of two or more thereof, preferably microcrystalline cellulose.

The binder will be optionally present in the pharmaceutical compositions of the invention in an amount within the range from about 0 to about 20% weight, preferably from about 1 to about 10% by weight of the composition. Examples of binders suitable for use herein include, but are not limited to, hydroxypropyl cellulose, corn starch, pregelatinized starch, modified corn starch, polyvinyl pyrrolidone (PVP) (molecular weight ranging from about 5,000 to about 1,000,000, preferably about 40,000), hydroxypropylmethyl cellulose (HPMC), lactose, gum acacia, ethyl cellulose, cellulose acetate, as well as a wax binder such as carnauba wax, paraffin, spermaceti, polyethylenes or microcrystalline wax, as well as other conventional binding agent and/or mixtures by two or more thereof, preferably hydroxypropyl cellulose.

The disintegrant will be optionally present in the pharmaceutical composition of the invention in an amount within the range from about 0 to about 20% by weight, preferably from about 0.25 to about 15% by weight of the composition. Examples of disintegrants suitable for use herein include, but are not limited to, croscarmellose sodium, crospovidone, starch, potato starch, pregelatinized starch, corn starch, sodium starch glycolate, microcrystalline cellulose, low substituted hydroxypropyl cellulose or other known disintegrant, preferably croscarmellose sodium.

The lubricant will be optimally present in the pharmaceutical composition of the invention in an amount within the range from about 0.1 to about 5% by weight, preferably from about 0.2 to about 2% by weight of the composition. Examples of tableting lubricants suitable for use herein include, but are not limited to, magnesium stearate, zinc stearate, calcium stearate, talc, carnauba wax, stearic acid, palmitic acid, sodium stearyl fumarate or hydrogenated vegetable oils and fats, or other known tableting lubricants, and/or mixtures of two or more thereof, preferably magnesium stearate.

The coating layer formulation (also referred to as the first coating layer) may be prepared as described hereinbefore and will contain medicament, coating layer polymer such as hydroxypropylmethyl cellulose, polyvinyl acetate, polyvinyl alcohol, ethyl cellulose, methacrylic polymers or hydroxypropyl cellulose, preferably hydroxypropylmethyl cellulose or polyvinyl alcohol. The coating layer may also include a plasticizer such as triacetin, diethyl phthalate, tributyl sebacate or polyethylene glycol, preferably triacetin; and an anti-adherent or glidant such as talc or opacifying agent such as titanium dioxide, fumed silica or magnesium stearate, preferably titanium dioxide.

The second coating layer may be similar in composition to the first coating layer although it will preferably not include medicament, and at least not the medicament present in the first coating layer.

The first coating layer will be formed of coating polymer in an amount within the range from about 10 to about 95%, preferably from about 30 to about 88% by weight of the coating layer, and medicament in an amount within the range from about 5 to about 90%, preferably from about 14 to about 70% by weight of the 5 coating layer, optionally plasticizer in an amount within the range from about 5 to about 30%, preferably from about 8 to about 9% by weight of the coating layer, and opacifying agent in an amount within the range for about 20 to about 40%, preferably from about 30 to about 35% by weight of the coating layer and optionally, coloring agent such as red, yellow or a combination red and yellow iron oxides in 0.1 to 3%, preferably 0.5 to 2%.

Preferred coated tablet formulations in accordance with the invention are set out below.

| | Possible Range % | |
|---|---|---|
| | by weight of | Preferred Range |
| | tablet core/mg | % by weight/mg |
| | (for 200 mg | (for 200 mg |
| | tablet core) | tablet core) |
| Material | | |
| Tablet Core | | |
| Bulking Agent | 2 to 95%/ | 10 to 85%/ |
| | 4 to 190 mg | 20 to 170 mg |
| Lactose | 0 to 95%/ | 20 to 75%/ |
| | 0 to 190 mg | 40 to 150 mg |
| Microcrystalline | 0 to 95%/ | 20 to 75%/ |
| cellulose | 0 to 190 mg | 40 to 150 mg |
| Disintegrant | 0 to 20%/ | 0.25 to 15%/ |
| | 0 to 40 mg | 0.5 to 30 mg |
| Croscarmellose | 1 to 20%/ | 2 to 10%/ |
| sodium | 0.5 to 40 mg | 4 to 20 mg |
| Lubricant | 0 to 4%/ | 0.2 to 2%/ |
| | 0 to 8 mg | 0.4 to 4 mg |
| Magnesium | 0.1 to 4%/ | 0.2 to 2%/ |
| Stearate | 0.2 to 8 mg | 0.4 to 4 mg |
| First Film | % by weight | % by weight |
| Coating | of film coating/ | of film coating/ |
| | mg (regardless | mg (regardless |
| | of weight of | of weight of |
| | tablet core) | tablet core) |
| Medicament | | |
| PPAR α/γ | 5 to 90%/ | 14 to 67%/ |
| dual agonist | 0.1 to 200 mg | 0.2 to 50 mg |
| Coating polymer, | 10 to 95%/ | 30 to 88%/ |
| and optional | 15 to 190 mg | 3 to 100 mg |
| plasticizer, | | |
| glidants and color | | |
| Second Film | % by weight/ | % by weight |
| Coating | mg with | of film coating/ |
| | second film | mg regardless |
| | coating | of weight of |
| | | tablet placebo |
| Coating polymer, | 100%/ | 100%/ |
| and optional | 1 to 25 mg | 2 to 15 mg |
| glidants and color | | |

The following Examples represent preferred embodiments of the invention.

### EXAMPLES

### Example 1

Film coated tablets, 0.5 mg, 1 mg, 2 mg, 4 mg, 8 mg and 10 mg, having the PPAR α/γ dual agonist Compound A (peliglitazar) coated thereon were prepared as follows.

Tablet cores for film coating having the following composition were prepared as follows.

TABLE 1
Composition of Tablet Core for film coating

| | Amount, mg/tablet | |
|---|---|---|
| Ingredient | (% w/w in tablet) | |
| Lactose Monohydrate, NF | 99 | (49.5%) |
| Microcrystalline Cellulose, NF | 90 | (45.0%) |
| Croscarmellose Sodium, NF | 10 | (5.0%) |
| Magnesium Stearate, NF | 1 | (0.5%) |
| Total | 200 | (100.0%) |

Lactose monohydrate, microcrystalline cellulose, and croscarmellose sodium were blended in an appropriate mixer, then lubricated by blending with magnesium stearate using a Turbula or an appropriate mixer. The lubricated blend was compressed into 200 mg or suitable weight tablet cores using a conventional tablet press.

TABLE 2
Composition of film coating suspension and film weight for PPAR α/γ dual
agonist film coated tablets, 0.5, 1, 2, 4, 8, and 10 mg

| | Strength | | | | | | | | | | | |
|---|---|---|---|---|---|---|---|---|---|---|---|---|
| | 0.5 mg | | 1 mg | | 2 mg | | 4 mg | | 8 mg | | 10 mg | |
| Ingredients | Amount, mg/tablet (%, w/w in suspension) | | | | | | | | | | | |
| Suspension for the | | | | | | | | | | | | |
| first film coat | | | | | | | | | | | | |
| PPAR α/γ dual agonist | 0.5 | (1.5%) | 1.0 | (1.5%) | 2.0 | (2.6%) | 4.0 | (4.0%) | 8 | (5.6%) | 10 | (6.06%) |
| Compound A (peliglitazar) | | | | | | | | | | | | |
| Opadry ® orange | 3.0 | (9.0%) | 6.0 | (9.0%) | 5.0 | (6.5%) | 5.0 | (5.0%) | 5 | (3.5%) | 5 | (3.03%) |
| Water* | 30 | (8.95%) | 60 | (89.5%) | 70 | (90.9%) | 91 | (91.0%) | 130 | (90.9%) | 150 | (90.9%) |
| Tablet weight gain after | 3.5 | | 7.0 | | 7.0 | | 9.0 | | 13.0 | | 15.0 | |
| the first film coat | | | | | | | | | | | | |

| Suspension for the | |
|---|---|
| second film coat | |
| Opadry ® orange | 5 (10.0%) |
| Water* | 45 (90.0%) |
| Tablet weight gain after the | 5.0 |
| second film coat | |

*This is used for processing only and is removed during the film coating process.

A suspension for a first film coat having the composition set out in Table 2 above was prepared as follows.

The PPAR α/γ dual agonist was mixed with Opadry® orange (that is hydroxypropylmethyl cellulose), and water employing a mechanical mixer. The resulting mixture was passed through a homogenizer to reduce drug particle size and to form a uniform suspension containing drug.

Alternatively, the suspension can also be prepared as follows. The PPAR α/γ dual agonist is added into water and passed through a homogenizer to reduce drug particle size. Then Opadry orange is mixed in using a mechanical mixer or homogenizer.

A first film coat was applied over the tablet cores using the above suspension until the target weight gains for the first film coat shown in Table 2 were obtained.

After the first film coat was dry, a suspension of a second film coat formulation having the composition set out in Table 2 was applied onto the film coated tablets until an additional weight gain of approximately 5 mg/tablet was obtained.

Stability of the film coated tablets was evaluated by packaging tablets (1 mg potency) in HDPE bottles with cotton coil, desiccant, heat induction seal and storing the bottles for six months at various storage conditions, namely at 5° C.; at 30° C./60% relative humidity (RH) at 40° C./75% RH, and at 40° C./75% RH open. Tablets were also exposed to 40° C./75% RH in an open petri dish.

The resulting film coated tablets of the invention were found to have superior stability over tablets of similar composition coating medicament in the tablet and not in a coating therefor, produced by conventional wet granulation.

The results for a 1 mg tablet are shown in the table set out below.

TABLE 3
Six month stability data of 1 mg potency spray-coated tablets
of the invention and 1 mg wet granulated tablets

| | | PPAR | | | | | |
|---|---|---|---|---|---|---|---|
| | | dual α/γ | | | | | |
| | | agonist | Base catalyzed | | Acid catalyzed | | |
| | | compound | degradants | | degradants | | |
| | Storage | % | 4-methoxy | Degradant | Glycine | Benzylic | Total, |
| Formulation | Condition | Original | phenol | Compound A′ | Carbamate | Alcohol | % I.I |
| Spray | 5° C. | 100 | — | — | — | — | 1.1 |
| Coated | 30° C./60% RH | 100 | — | — | — | — | 1.5 |
| Tablets of | | | | | | | |
| the Example | 40° C./75% RH | 99 | 0.10 | 0.15 | — | — | 1.4 |
| | 40° C./75% RH open | 99 | 0.10 | 0.46 | — | — | 2.4 |
| Wet | 5° C. | 103 | — | — | — | — | 0.5 |
| Granulation | 30° C./60% RH | 102 | 0.16 | 0.24 | — | — | 1.0 |
| Tablets | | | | | | | |
| | 40° C./75% RH | 98 | 0.61 | 1.42 | — | — | 2.7 |
| | 40° C./75% RH open | 86 | 0.75 | 5.31 | 0.06 | 0.06 | 8.4 |

It is theorized that high drug to excipient ratio in the polymer coating attributes to the superior stability of the coated tablets of the invention (1 mg drug in 10 mg polymer coating) over conventional tablets (1 mg drug in 200 mg tablets).

The batch parameters and results for the peliglitazar tablets are shown in the table set out below.

TABLE 4
Coating Parameters and Batch Results for Peliglitazar Film Coated Tablets

| | Strength | | | | | |
|---|---|---|---|---|---|---|
| | 0.5 mg | 1 mg | 2 mg | 4 mg | 8 mg | 10 mg |
| Lot Number | 56678-168 | 56678-143 | 56777-106 | 56777-106 | 56678-164 | 56678-049 |
| Batch Size, kg | 14 | 14 | 14 | 14 | 17 | 0.6 |
| Pan Speed, rpm | 18 | 20 | 17 | 17 | 18 | 25 |
| Suspension Flow | 20 | 20 | 20 | 30 | 35 | 5.5 |
| Rate, mg/mL | | | | | | |
| Nozzle Size, mm | 0.42 | 0.42 | 0.42 | 0.42 | 0.42 | 1 |
| Atomization | 39 | 39 | 39 | 45 | 45 | 11 |
| Pressure, psi | | | | | | |
| Coating Time | 3.0 | 4.5 | 4.5 | 5 | 5.25 | 2.2 |
| (1^{st} film), hours | | | | | | |
| Tablet Potency, | 102 | 102 | 106 | 104 | 97 | 100.5 |
| % label (mg) | (0.51) | (1.02) | (2.12) | (4.16) | (7.76) | (10.05) |
| RSD | 1.8% | 2.8% | 2.5% | 2.7% | 2.2% | 1.5% |

### Example 2

Film coated tablets, 1 mg and 8 mg, having the PPAR α/γ dual agonist Compound B (muraglitazar) coated thereon were prepared as follows.

Tablet cores for film coating having the following composition were prepared as follows.

TABLE 5
Composition of Tablet Core for film coating

| | Amount, mg/tablet | | | |
|---|---|---|---|---|
| | (% w/w in tablet) | | | |
| | Used in 1 | | Used in 8 | |
| Ingredient | mg tablet | | mg tablet | |
| Lactose Monohydrate, NF | 109 | (54.5%) | 99 | (49.5%) |
| Microcrystalline Cellulose, NF | 80 | (40%) | 90 | (45.0%) |
| Croscarmellose Sodium, NF | 10 | (5%) | 10 | (5.0%) |
| Magnesium Stearate, NF | 1 | (0.5%) | 1 | (0.5%) |
| Total | 200 | (100%) | 200 | (100.0%) |

Lactose monohydrate, microcrystalline cellulose, and croscarmellose sodium were blended in an appropriate mixer, then lubricated by blending with magnesium stearate using a Turbula or an appropriate mixer. The lubricated blend was compressed into 200 mg or suitable weight tablet cores using a conventional tablet press.

TABLE 6
Composition of film coating suspension and film weight for PPAR α/γ
dual agonist (muraglitazar) film coated tablets, 1 and 8 mg

Strength

| | |
|---|---|
| 1 mg | 8 mg |

| | |
|---|---|
| | Amount, mg/tablet (%, |
| Ingredients | w/w in suspension) |

| | | | | |
|---|---|---|---|---|
| Suspension for the | | | | |
| first film coat | | | | |
| PPAR α/γ dual | 1.0 | (1.6%) | 8 | (6.0%) |
| agonist Compound B | | | | |
| (muraglitazar) | | | | |
| Opadry ® orange | 6.0 | (9.6%) | 5 | (3.75%) |
| Water* | 55.5 | (88.8%) | 120 | (90.25%) |
| Tablet weight gain | 7.0 | | 13.0 | |
| after the first | | | | |
| film coat | | | | |

| | |
|---|---|
| Suspension for the | |
| second film coat | |
| Opadry ® orange | 5 (10.0%) |
| Water* | 45 (90.0%) |
| Tablet weight gain | 5.0 |
| after the second | |
| film coat | |

*This is used for processing only and is removed during the film coating process.

A suspension for a first film coat having the composition set out in Table 5 above was prepared as follows.

The PPAR α/γ dual agonist was mixed with Opadry® orange (that is hydroxypropylmethyl cellulose), and water employing a mechanical mixer. The resulting mixture was passed through a homogenizer to reduce drug particle size and to form a uniform suspension containing drug.

Alternatively, the suspension can also be prepared as follows. The PPAR α/γ dual agonist is added into water and passed through a homogenizer to reduce drug particle size. Then Opadry orange is mixed in using a mechanical mixer or homogenizer.

A first film coat was applied over the tablet cores using the above suspension until the target weight gains for the first film coat shown in Table 6 were obtained.

After the first film coat was dry, a suspension of a second film coat formulation having the composition set out in Table 5 can be applied onto the film coated tablets until an additional weight gain of approximately 5 mg/tablet was obtained.

The batch parameters and results for 1 and 8 mg tablets are shown in the table set out below.

TABLE 7
Coating parameters and batch results:

| | |
|---|---|
| 1 mg | 8 mg |

| Lot number | 56678-139 | | 53777-069 | |
|---|---|---|---|---|
| Batch Size | 17 | kg | 11 | kg |
| Pan speed | 15 | rpm | 20 | rpm |
| Suspension flow Rate | 25 | mL/min | 30 | mL/min |
| Nozzle size | 0.7 | mm | 0.7 | |
| Atomization Pressure | 44 psi (3 bar) | | 44 psi (3 bar) | |
| Coating Time (hrs) | 3:25 | | 5:15 | |
| Average Tablet Potency | 1.008 | mg | 8.4 | mg |

| | | |
|---|---|---|
| RSD | 4.6% | 3.5% |

1. A coated tablet comprising a tablet core and at least one coating layer coated thereon, which coating layer comprises a medicament and at least one coating polymer formulation.

2. The coated tablet as defined in claim 1 wherein the medicament is subject to base catalyzed degradation and/or acid catalyzed degradation.

3. The coated tablet as defined in claim 1 wherein the medicament is a PPAR α/γ dual agonist.

4. The coated tablet as defined in claim 3 wherein the medicament is peliglitazar which has the structure

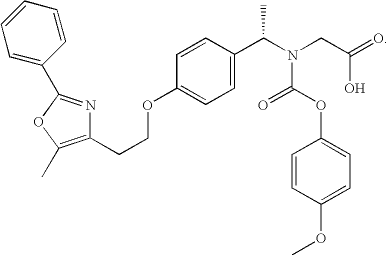

5. The coated tablet as defined in claim 3 wherein the medicament is muraglitazar which has the structure

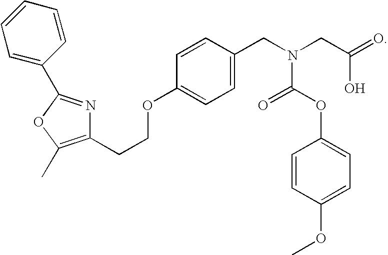

6. The coated tablet as defined in claim 1 wherein said coating layer is a spray dried coating.

7. The coated tablet as defined in claim 1 wherein said coating layer is formed of a coating polymer formulation comprising a hydroxypropylmethyl cellulose based polymer, polyvinyl alcohol, polyvinyl acetate, ethyl cellulose, methacrylic polymer, or hydroxypropyl cellulose.

8. The coated tablet as defined in claim 1 wherein the coating layer is formed of a coating polymer formulation comprising a hydroxypropylmethyl cellulose based polymer.

9. The coated tablet as defined in claim 1 wherein said coating layer comprises hydroxypropylmethyl cellulose, titanium oxide and triacetin.

10. The coated tablet as defined in claim 1 wherein said coating layer is comprised of from about 14 to about 67% by weight medicament and from about 30 to about 88% by weight coating polymer.

11. The coated tablet as defined in claim 1 wherein the coating polymer formulation is at least about 5 mg with a 200 mg tablet core, and the medicament is at least about 0.1% based on the weight of the tablet core or 0.2 mg.

12. The coated tablet as defined in claim 1 further including a second coating layer disposed on said coating layer.

13. The coated tablet as defined in claim 12 wherein said second coating layer comprises a hydroxypropylmethyl cellulose based polymer or polyvinyl acetate, polyvinyl alcohol, ethyl cellulose, methacrylic polymer, or hydroxypropyl cellulose.

14. The coated tablet as defined in claim 13 wherein the second coating layer is comprised of hydroxypropylmethyl cellulose based polymer.

15. The coated tablet as defined in claim 12 wherein the coating layer and the second coating layer each indicates substantially the same hydroxypropylmethyl cellulose based polymer.

16. The coated tablet as defined in claim 1 comprising from about 0.1 to about 70% by weight medicament, based on the weight of the finished tablet.

17. The coated tablet as defined in claim 15 wherein the medicament is peliglitazar which has the structure

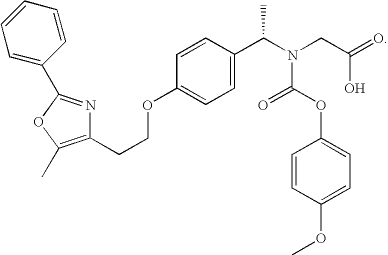

18. The coated tablet as defined in claim 15 wherein the medicament is muraglitazar which has the structure

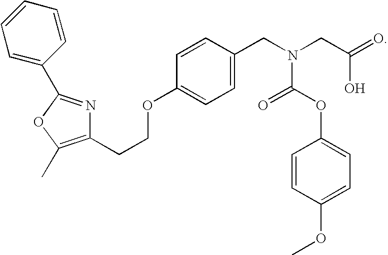

19. The coated tablet as defined in claim 1 wherein the medicament is present in an amount within the range from about 0.1 to about 25 mg and the coating polymer is present in an amount within the range from about 1 to about 50 mg, and optionally including a second coating layer disposed over the coating layer, said second coating layer being present in an amount within the range from about 1 to about 50 mg.

20. The coated tablet as defined in claim 1 wherein the tablet core is comprised of one or more fillers, optionally one or more binders, and one or more disintegrants and one or more tableting lubricants.

21. The coated tablet as defined in claim 17 wherein the tablet core is comprised of microcrystalline cellulose, lactose monohydrate, croscarmellose sodium and magnesium stearate.

22. The coated tablet as defined in claim 1 having the following composition:

| | % by weight of | |
|---|---|---|
| | tablet core | |
| Tablet Core | | |
| Microcrystalline cellulose | 20 to 75% | by weight |
| Lactose monohydrate | 20 to 75% | by weight |
| Croscarmellose sodium | 2 to 10% | by weight |
| Magnesium stearate | 0.2 to 2% | by weight |
| First coating layer | | |
| Medicament | 0.2 to 50 | mg |
| Hydroxypropylmethyl cellulose or | 20 to 180 | mg |
| Polyvinyl alcohol based coating material | | |
| Second coating layer | | |
| Hydroxypropylmethyl cellulose or | 2 to 15 | mg |
| Polyvinyl alcohol based coating material | | |

wherein the medicament has the structure

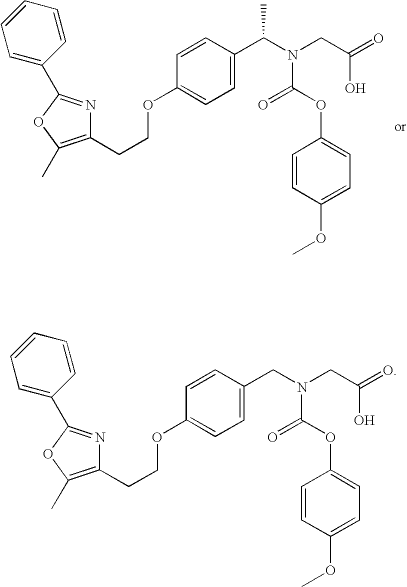

23. The coated tablet as defined in claim 22 where for a 10 mg potency the coating layer is comprised of 10 mg medicament and 5 mg polymer based coating and for a 1 mg potency the coating layer is comprised of 1 mg medicament and 6 mg polymer based coating.

24. A coated tablet comprising:
a) a tablet core which comprises one or more excipients and optionally one or more active ingredients and optionally one or more medicaments;
b) at least one coating layer coated on the tablet core, which layer comprises at least one medicament and at least one coating polymer formulation; and
c) optionally a second coating layer disposed on the coating layer of b), said second coating layer comprising a coating polymer formulation.

25. The coated tablet as defined in claim 1 having the following composition:
Tablet Core

| | Amount, mg/tablet | |
|---|---|---|
| Ingredient | (% w/w in tablet) | |
| Lactose Monohydrate, NF | 99 | (49.5%) |
| Microcrystalline Cellulose, NF | 90 | (45.0%) |
| Croscarmellose Sodium, NF | 10 | (5.0%) |
| Magnesium Stearate, NF | 1 | (0.5%) |
| Total | 200 | (100.0%) |

Lactose monohydrate, microcrystalline cellulose, and croscarmellose sodium were blended in an appropriate mixer, then lubricated by blending with magnesium stearate using a Turbula or an appropriate mixer. The lubricated blend was compressed into 200 mg or suitable weight tablet cores using a conventional tablet press.
Composition of film coating layers and film weight for peliglitazar film coated tablets, 0.5, 1, 2, 4, 8, and 10 mg

| | Strength | | | | | | | | | | | |
|---|---|---|---|---|---|---|---|---|---|---|---|---|
| | 0.5 mg | | 1 mg | | 2 mg | | 4 mg | | 8 mg | | 10 mg | |
| Ingredients | Amount, mg/tablet (%, w/w in suspension) | | | | | | | | | | | |
| First film coating layer | | | | | | | | | | | | |
| PPAR α/γ dual agonist | 0.5 | (1.5%) | 1.0 | (1.5%) | 2.0 | (2.6%) | 4.0 | (4.0%) | 8 | (5.6%) | 10 | (6.06%) |
| peliglitazar - Compound A | | | | | | | | | | | | |
| Hydroxypropylmethyl cellulose | 3.0 | (9.0%) | 6.0 | (9.0%) | 5.0 | (6.5%) | 5.0 | (5.0%) | 5 | (3.5%) | 5 | (3.03%) |
| Water* | 30 | (8.95%) | 60 | (89.5%) | 70 | (90.9%) | 91 | (91.0%) | 130 | (90.9%) | 150 | (90.9%) |
| Tablet weight gain after the | 3.5 | | 7.0 | | 7.0 | | 9.0 | | 13.0 | | 15.0 | |
| first film coating layer | | | | | | | | | | | | |
| Second film coating layer | |
|---|---|
| Hydroxypropylmethyl cellulose | 5 (10.0%) |
| Water* | 45 (90.0%) |
| Tablet weight gain after the | 5.0 |
| second film coating layer | |

*Water is used for processing only and is removed during the film coating process.

26. The coated tablet as defined in claim 1 having the following composition:
Tablet Core

| | Amount, mg/tablet | | | |
|---|---|---|---|---|
| | (% w/w in tablet) | | | |
| | Used in 1 | | Used in 8 | |
| Ingredient | mg tablet | | mg tablet | |
| Lactose Monohydrate, NF | 109 | (54.5%) | 99 | (49.5%) |
| Microcrystalline Cellulose, NF | 80 | (40%) | 90 | (45.0%) |
| Croscarmellose Sodium, NF | 10 | (5%) | 10 | (5.0%) |
| Magnesium Stearate, NF | 1 | (0.5%) | 1 | (0.5%) |
| Total | 200 | (100%) | 200 | (100.0%) |

Composition of Film Coating Layers and Film Weight For Muraglitazar Film Coated Tablets, 1 and 8 mg
Strength

| | |
|---|---|
| 1 mg | 8 mg |
| | |
|---|---|
| | Amount, mg/tablet (%, |
| Ingredients | w/w in suspension) |
| | | | | |
|---|---|---|---|---|
| First film coating layer | | | | |
| PPAR α/γ dual agonist | 1.0 | (1.6%) | 8 | (6.0%) |
| muraglitazar - Compound B | | | | |
| Hydroxypropylmethyl | 6.0 | (9.6%) | 5 | (3.75%) |
| cellulose | | | | |
| Water* | 55.5 | (88.8%) | 120 | (90.25%) |
| Tablet weight gain after | 7.0 | | 13.0 | |
| the first film coating | | | | |
| layer | | | | |
| | |
|---|---|
| Second film coating layer | |
| Hydroxypropylmethyl | 5 (10.0%) |
| cellulose | |
| Water* | 45 (90.0%) |
| Tablet weight gain after | 5.0 |
| the second film coating | |
| layer | |

*Water is used for processing only and is removed during the film coating process.

27. A method for preparing a coated tablet comprising a tablet core and at least one coating layer coated thereon, which coating layer comprises a medicament and at least one coating polymer, which method comprises applying a coating layer to one or more tablet cores, and drying the coated tablets.

28. The method as defined in claim 27 wherein the coating layer is applied as a suspension of the coating polymer.

29. The method as defined in claim 27 including the steps of applying a second coating layer over the coating layer and drying the so-coated tablet cores.
